(19) United States
(12) Patent Application Publication
(10) Pub. No.: US 2013/0137975 A1
(43) Pub. Date: May 30, 2013

(54) PET IMAGING

(71) Applicants: Joseph E. Bugaj, Longmont, CO (US); Henry C. Manning, Nashville, TN (US); Eliot T. McKinley, Nashville, TN (US); Robert C. Wild, Carmel, IN (US)

(72) Inventors: Joseph E. Bugaj, Longmont, CO (US); Henry C. Manning, Nashville, TN (US); Eliot T. McKinley, Nashville, TN (US); Robert C. Wild, Carmel, IN (US)

(73) Assignee: OSI PHARMACEUTICALS, LLC, Farmingdale, NY (US)

(21) Appl. No.: 13/576,975

(22) PCT Filed: Feb. 9, 2011
(86) PCT No.: PCT/US2011/024121

§ 371 Date: Oct. 10, 2012

Related U.S. Application Data
(60) Provisional application No. 61/302,886, filed on Feb. 9, 2010.

(51) Int. Cl. A61B 6/03; A61K 51/00; A61B 6/00

(52) U.S. Cl. CPC A61B 6/037; A61B 6/481; A61K 51/00; A61B 6/502; A61B 6/501
USPC 600/426

(57) ABSTRACT

A method for determining whether to administer a therapeutically effective amount of a receptor tyrosine kinase-inhibiting drug for tumor treatment, by determining a glucose uptake response in the tumor of a mammal. ¹⁸FDG-PET (2-deoxy-2-[¹⁸F]fluoro-D-glucose positron emission tomography imaging) is used as a predictive, non-invasive, pharmacodynamic biomarker of response following administration of drug.

### CROSS-REFERENCE TO RELATED APPLICATIONS

This application claims the benefit of U.S. Provisional Application No. 61/302,886, filed Feb. 9, 2010, which is herein incorporated by reference in its entirety.

### FIELD OF THE INVENTION

The present invention generally relates in some aspects to medical diagnostics, positron emission tomography (PET) imaging, and anti-cancer therapy.

### BACKGROUND OF THE INVENTION

Positron emission tomography (PET) is a well-known imaging technique used for producing an image or map of functional processes in the body. The technique is particularly useful in imaging of tumors and other cancerous tissue. The technique involves the administration to a subject of a positron-emitting radionuclide tracer followed by detection of the positron emission (annihilation) events in the body. The radionuclide tracer is typically composed of a targeting molecule having incorporated therein one or more types of positron-emitting radionuclides, such as fluorine-18, oxygen-15, nitrogen-13, carbon-11, and iodine-124.

PET has particularly been used for staging disease and/or monitoring treatment effects of a drug in a mammal. In this regard, PET has conventionally been used to evaluate treatment response after long-term drug therapy (typically, after a treatment regimen lasting days, weeks, or months). However, this practice has several drawbacks and limitations. For example, the conventional method can unnecessarily subject a cancer patient to a full regimen of an anti-cancer drug before knowing whether the drug is effective for the patient (i.e., whether the patient is receptive to the drug or whether the administered dose is effective or optimal). After a full regimen is complete, and it is determined that the treatment was not effective or optimal, the patient has lost considerable time in treating the cancer. In addition, the patient may suffer mild to serious side effects that the patient would not have been subjected to had the lack of efficacy of the drug been known in advance and the treatment modified.

Clinical studies have shown that ¹⁸F-FDG (2-deoxy-2-[¹⁸F]fluoro-D-glucose) uptake of human tumors can be measured by FDG-PET with high reproducibility. See, e.g., Weber et al., “Use of PET for Monitoring Cancer Therapy and for Predicting Outcome*”, The Journal of Nuclear Medicine, 46, 983-995, (2005).

Accordingly, there is a need for a non-invasive method that could provide a real-time, quantitative evaluation (e.g., directly following a first therapeutic or sub-therapeutic dose of an anti-cancer drug) on the PD (pharmacodynamic) effects of the drug. The information provided can be used, for example, to determine whether the drug is binding the intended target of interest and eliciting a pharmacological response, determine whether the drug is suitable or potentially efficacious in treating the cancer in a particular patient, or determine an appropriate pharmacologically effective dose and dosage regimen for a patient. If the tissue is found to be cancerous, the method, when combined with molecular targeted therapies, can also provide information on the type and predominant molecular pathways responsible for sustainment of the cancer. Such a method would provide an improved treatment for cancer patients by determining in the early or planning phases of treatment an effective or optimal course of treatment. See, e.g., Song et al., “Changes in ¹⁸F-FDG Uptake Within Minutes After Chemotherapy in a Rabbit VX2 Tumor Model”, The Journal of Nuclear Medicine, 49, 303-309 (2008).

Glucose metabolic activity closely reflects response to gefitinib therapy. See, e.g., Su et al., “Monitoring Tumor Glucose Utilization by Positron Emission Tomography for the Prediction of Treatment Response to Epidermal Growth Factor Receptor Kinase Inhibitors”, Clinical Cancer Research, 5659 12(19), (2006). FDG-PET may be a valuable clinical predictor, early in the course of treatment, for therapeutic responses to EGFR kinase inhibitors. See id. at 5659. Furthermore, while clinical studies with ¹⁸F-FDG are promising there remains a need for additional safe and well tolerated radioactive labeled compounds. For example, 1-deoxy-1-[¹⁸F]-fluoro-D-fructose is being investigated. See, e.g., US 2009/0175787 (published Jul. 9, 2009).

It is recognized that RTK-inhibiting (receptor tyrosine kinase-inhibiting) drugs are useful as selective inhibitors of the growth of mammalian cancer cells. For example, OSI-906 is a novel, potent, selective and orally bioavailable dual IGF-1R/IR kinase inhibitor in Phase III clinical development in various cancers and tumor types. US 2006/0235031 (published Oct. 19, 2006) describes a class of bicyclic ring substituted protein kinase inhibitors, including Example 31 thereof, which corresponds to the IGF-1R inhibitor known as OSI-906.

### SUMMARY OF THE INVENTION

In some aspects, the invention is directed to a method for determining whether to administer a therapeutically effective amount of a drug for tumor treatment. In some aspects, the invention employs a radio labeled monosaccharide (e.g., ¹⁸F-FDG) as a predictive, non-invasive, PD biomarker of response following administration of a drug. In some aspects, the invention is used, for example, to assess the responsiveness of a particular patient's cancer to said drug before subjecting the patient to a full dosage regimen. In some aspects, the invention described herein significantly improves the quality of anti-cancer drug therapies by, for example, determining in the early or planning stage of treatment an effective or optimal course of treatment. In some aspects, the invention includes a method for determining whether to administer a therapeutically effective amount of OSI-906 (cis-3-[8-amino-1-(2-phenyl-quinolin-7-yl)-imidazo[1,5-a]pyrazin-3-yl]-1-methylcyclobutanol) represented by the following formula:

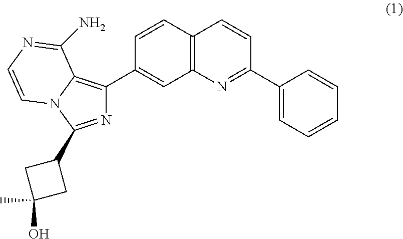

### BRIEF DESCRIPTION OF THE DRAWINGS

The features and aspects of the present invention will be better understood by reference to the following figures.

FIG. 1. Histogram graph showing dose-dependent inhibition of ³H-2-deoxyglucose uptake in H292 cells in vitro after 30-minute treatment with OSI-906.

FIG. 2. Normalized histogram showing ³H-2-deoxyglucose uptake in H292 cells in vitro at 0.0 μM, 2.5 μM, 5.0 μM, and 7.5 μM concentrations of OSI-906 from left to right in each set of four shaded bars, measured at 45 (darkest bars), 30 (lighter bars) and 5 (lightest bars) minutes post treatment. Data normalized to percentage (%) of control.

FIG. 3. Histogram graph showing ³H-2-deoxyglucose uptake in H292 cells in vitro treated with cytochalasin-B and OSI-906 at different concentrations measured at 30 minutes post treatment.

FIG. 4. ¹⁸F-FDG-PET images of representative H292 xenografts and H441 xenografts in vivo at 0, 2, 4 and 24 hours post drug treatment with 60 mg/kg of OSI-906.

FIGS. 5 and 6. Charts showing ¹⁸F-FDG uptake in H292 tumors and H441 tumors as a function of drug treatment and time. ¹⁸F-FDG uptake was determined at 2, 4 and 24 hours post OSI-906 treatment and measured by FDG-PET SUV (% ID/g) compared to negative control- and vehicle control-treated mice.

FIG. 7. Inhibition of ¹⁸F-FDG uptake in H292 tumors in vivo at 2, 4, 8 and 24 hours post drug treatment with 60 mg/kg of OSI-906 versus vehicle treated controls. Uptake was quantified by scintillation counting, and calculated as % injected dose/gram of tumor tissue.

FIG. 8. Inhibition of phospho-IGF-1R (dark shading) and phospho-IR (lighter shading) in H292 tumor samples in vivo at 2, and 4 hours post treatment with 60 mg/kg OSI-906. Inhibition was calculated as % of untreated control.

FIGS. 9 and 10. Daily treatment of mice bearing NCI H292 xenografts with 60 mg/kg OSI-906 results in significant tumor growth inhibition (FIG. 9) compared to analogously treated vehicle controls. In contrast, NCI-H441 xenografts (FIG. 10) do not exhibit a difference in tumor growth when comparing OSI-906-treated and vehicle-treated cohorts.

FIG. 11. Western Blot analysis of H292 in vitro cell lysates subjected to increasing concentrations of OSI-906 (0.0 μM-30 μM) for phospho-AKT (pAKT) (top row), total AKT (second row), phospho-S6 (pS6) (third row), Total S6 (fourth row), and AS160 (bottom row). Samples were collected 30 minutes after drug treatment.

FIG. 12. Western blot of NCI-H292 cells following 30 minutes of exposure to OSI-906 shows target inhibition of pIGF-1R and pIR at all doses as well as inhibition of downstream targets pIGF-1R, pIR, pERK, pAKT, pS6, pIGF-1R and AS160.

FIG. 13. ³H-2-deoxyglucose uptake 30 minutes after OSI-906 treatment in NCI-H292 cells showed a dose dependent decrease in ³H-2-deoxyglucose uptake at higher doses of OSI-906 in the non responding NCI-H441 cells compared with the responding NCI H292 cells.

FIG. 14. In vivo Western blot of NCI-H292 tumor lysates at 4 and 24 hours shows inhibition of selected markers of altered glycolysis, pERK, pAKT, pS6 at 4 hours post dose that return to baseline levels by 24 hours.

FIG. 15. Western blot of NCI-H292 cells treated with 10 nM, 100 nM, 500 nM, 1 mM, and 5 mM OSI-906 show target inhibition over a 24 hour time course. All concentrations of OSI-906 induce a reduction in pIGF-1R at 2 hours, and inhibition remains through 24 hours in all but the lowest, 10 nM concentration.

FIG. 16. Histogram graph demonstrating rapid inhibition of ³H-2-deoxyglucose uptake in H292 cells in vitro after 30 minute treatment with OSI-906 or erlotinib at various concentrations.

FIGS. 17 and 18. RTK array analysis demonstrates strong target inhibition of both pIGF-1R and pIR in NCI-H292 tumor lysates at 2, 4 and 24 hours after a single 60 mg/kg treatment of OSI-906.

### DETAILED DESCRIPTION OF THE INVENTION

In some aspects, the invention is directed to a method for determining whether to administer a receptor tyrosine kinase-inhibiting drug for tumor treatment and evaluating the pharmacodynamic effects of an RTK-inhibiting drug by measuring inhibition of glucose uptake in a tumor of a mammal within the early or planning stages of determining a suitable drug treatment program. The subject can be any mammal (e.g., a mouse, cat, dog, horse, or ape), but the method is more typically directed to human subjects.

In some aspects, the invention is directed to a method for determining whether to administer to a mammal a receptor tyrosine kinase-inhibiting drug for tumor treatment by determining a glucose uptake response in a tumor in the mammal, the method comprising: (i) administering to said mammal a first amount of a radionuclide-labeled glucose analogue that is taken up into said tumor and functions as a tracer in positron emission tomography and thereby determining a first glucose uptake response; (ii) administering to said mammal a test dosage of the drug; (iii) administering to said mammal a second amount of the radionuclide-labeled glucose analogue for determining a second glucose uptake response; (iv) determining the second glucose uptake response of said tumor by positron emission tomography imaging of said tumor at about 0.5-24 hours after completion of (ii); and (v) determining whether to administer the drug based on its pharmacodynamic effect as determined by a degree of inhibition of the glucose uptake response observed by positron emission tomography imaging by comparing said first glucose uptake response and said second glucose uptake response, wherein an inhibitory effect on the glucose uptake response after administration of the test dosage indicates that the mammal is likely to benefit from treatment with the drug.

In some aspects, the invention is directed to a method for method for treating a tumor of a mammal with a receptor tyrosine kinase-inhibiting drug comprising: (A) initially determining if said mammal is likely to respond to treatment by: (i) administering to said mammal a first amount of a radionuclide-labeled glucose analogue that is taken up into said tumor and functions as a tracer in positron emission tomography and thereby determining a first glucose uptake response; (ii) administering to said mammal a test dosage of the drug; (iii) administering to said mammal a second amount of the radionuclide-labeled glucose analogue for determining a second glucose uptake response; (iv) determining the second glucose uptake response of said tumor by positron emission tomography imaging of said tumor at about 0.5-24 hours after completion of (ii); and (v) determining an inhibitory effect as a degree of inhibition of the glucose uptake response observed by positron emission tomography imaging by comparing said first glucose uptake response and said second glucose uptake response; and (B) administering to said mammal a therapeutically effective regimen comprising the drug if there is an inhibitory effect determined by (v).

In some aspects, the invention is directed to a method for method for evaluating pharmacodynamic effects of a receptor tyrosine kinase-inhibiting drug in inhibiting glucose uptake in a tumor of a mammal, the method comprising: (i) administering to said mammal a dosage of said drug; (ii) administering to said mammal a radionuclide-labeled glucose analogue; (iii) determining a glucose uptake response of said tumor by positron emission tomography imaging of said tumor at about 0.5-24 hours after completion of (i); and (iv) determining a degree of inhibition of the glucose uptake response observed by positron emission tomography imaging.

In some aspects, the invention is directed to said method, whereby said determining of (iii) is carried out at about 0.5 to 12 hours after completion of (i).

In some aspects, the invention is directed to said method, whereby said determining of (iv) is carried out at about 0.5 to 12 hours after completion of (ii).

In some aspects, the invention is directed to said method, whereby said determining of (iii) is carried out at about 0.5 to 4 hours after completion of (i).

In some aspects, the invention is directed to said method, whereby said determining of (iv) is carried out at about 0.5 to 4 hours after completion of (ii).

In some aspects, the invention is directed to said method, whereby said determining of (iii) is carried out at about 0.5 to 2 hours after completion of (i).

In some aspects, the invention is directed to said method, whereby said determining of (iv) is carried out at about 0.5 to 2 hours after completion of (ii).

In some aspects, the invention is directed to said method, whereby said determining of (iii) is carried out at about 0.5 to 1 hour after completion of (i).

In some aspects, the invention is directed to said method, whereby said determining of (iv) is carried out at about 0.5 to 1 hour after completion of (ii).

In some aspects, the invention is directed to said method, whereby said administration of (iii) is carried out before, simultaneously, or after (ii).

In some aspects, the invention is directed to said method, whereby said administration of (iii) is carried out at about 15 to 120 minutes before (ii).

In some aspects, the invention is directed to said method, whereby said administration of (iii) is carried out simultaneously with (ii).

In some aspects, the invention is directed to said method, whereby said administration of (iii) is carried out at about 15 to 120 minutes after (ii).

In some aspects, the invention is directed to said method, whereby said method is carried out once or multiple times.

In some aspects, the invention is directed to said method, whereby (ii) is carried out once and (iv) is carried out multiple times.

In some aspects, the invention is directed to said method, whereby said test dosage is a sub-therapeutic or therapeutic dose.

In some aspects, the invention is directed to said method, whereby said glucose uptake is expressed as a SUV.

In some aspects, the invention is directed to said method, whereby said inhibitory effect is about 20% to 50%.

In some aspects, the invention is directed to said method, whereby said method further comprises determining the efficacy of said drug in inhibiting target and downstream pathways leading to inhibition of metabolic activity, resulting in inhibition of growth or sustainment of said tumor.

In some aspects, the invention is directed to said method, whereby said method further comprises determining the responsiveness of said mammal to said drug based on said degree of inhibition of glucose uptake.

In some aspects, the invention is directed to said method, whereby said method further comprises determining a suitable dosage or dosage range of said for said mammal based on said degree of inhibition of glucose uptake.

In some aspects, the invention is directed to said method, whereby said method further comprises administering to said mammal varying concentrations of said drug to determine an optimal dosage or dosage range.

In some aspects, the invention is directed to said method, whereby said method further comprises optimization of an administration schedule of said drug for said mammal based on said degree of inhibition of glucose uptake.

In some aspects, the invention is directed to said method, whereby said method further comprises determining a maximum efficacious dose or biologically efficacious dose of said drug for said mammal based on said degree of inhibition of glucose uptake.

In some aspects, the invention is directed to said method, whereby said method further comprises determining a dosage schedule by administering to said mammal a single dose of said drug, and monitoring glucose uptake, by continuous imaging or imaging at several time points within 24 hours after completion of (iv).

In some aspects, the invention is directed to said method, whereby said method further comprises determining whether a level of glucose uptake in tissue of said mammal is indicative of said tissue being a tumor.

In some aspects, the invention is directed to said method, whereby said method further comprises determining a signaling pathway primarily responsible for driving said tumor, and from this, determining an appropriate treatment strategy for said mammal.

In some aspects, the invention is directed to said method, whereby said drug is a PI3K signaling pathway disruptor.

In some aspects, the invention is directed to said method, whereby said drug is erlotinib, sunitinib, sorafenib, imatinib, bosutinib, nilotinib, or dasatinib.

In some aspects, the invention is directed to said method, whereby said drug is an inhibitor of one or more of IR, IGF-1R or IR/IGF-1R heterodimer.

In some aspects, the invention is directed to said method, whereby said drug is BMS-754807, AG538, AG1024, AMG-479, NVP-AEW541, figitumumab, R-1507, cixutumumab, dalotuzumab, IGF-1R antibodies, or OSI-906.

In some aspects, the invention is directed to said method, whereby said drug is OSI-906.

In some aspects, the invention is directed to said method, whereby said tumor is in a lung, breast, prostate, bladder, colon, rectum, pancreas, ovary, stomach, head, neck, esophagus, liver, adrenal gland, or kidney of said mammal.

In some aspects, the invention is directed to said method, whereby said tumor is in a lung of said mammal.

In some aspects, the invention is directed to said method, whereby said tumor is NSCLC.

In some aspects, the invention is directed to said method, whereby said mammal is human.

In some aspects, the invention is directed to said method, whereby said radionuclide-labeled glucose analogue is ¹⁸F-FDG.

The RTK-inhibiting drug inhibits the activity of tyrosine kinase enzymes in animals, including humans, and can be useful in the treatment and/or prevention of various diseases and conditions such as hyperproliferative disorders such as cancer. In particular, compounds disclosed herein are inhibitors of IR, IGF-1R or IR/IGF-1R heterodimer.

The RTK-inhibiting drug can be any drug having an ability to target (e.g., inhibit, modulate, or disrupt) one or more receptor tyrosine kinases. The receptor tyrosine kinase being targeted can be, for example, a receptor of a tyrosine kinase growth factor, cytokine, or hormone. Some examples of receptor tyrosine kinases that may be targeted by the drug include the insulin receptor family, the EGF receptor family (i.e., as targeted by erlotinib), the PDGF receptor family, VEGF receptor family, and FGF receptor family, as well as those involved in the PI3K, Akt, or MAP kinase signaling pathways. Some examples of drugs considered herein are the tyrosine kinase inhibiting drugs, such as erlotinib (Tarceva®), sunitinib (Sutent®, SU11248), sorafenib (Nexavar®), imatinib (e.g., Gleevec®), bosutinib, nilotinib, and dasatinib.

The instant invention discloses those drugs that target the insulin receptor family. The insulin receptor family includes, for example, the insulin receptor (IR) and the insulin-like growth factor receptors. Some examples of insulin-like growth factor receptors include the insulin-like growth factor-1 receptor (IGF-1R) and insulin-like growth factor-2 receptor (IGF-2R). Some examples of drugs considered herein are the IR, and IGF-1R inhibiting drugs, such as BMS-754807, AG538, AG1024, AMG-479, NVP-AEW541, figitumumab (CP-751871), R-1507, cixutumumab (IMC-A12), dalotuzumab (MK-0646), and OSI-906. Other IGF-1R inhibitors are disclosed in US 2006/0235031.

OSI-906 is a selective tyrosine kinase inhibitor that exhibits similar biochemical potency against IGF-1R and IR and is greater than four orders of magnitude more selective for IGF-1R/IR compared to a wide number of other kinases, receptor and non-receptor, evaluated. Within a panel of 116 kinases, only IGF-1R and IR were inhibited greater than 50% by 1 μM OSI-906. Moreover, OSI-906 has demonstrated inhibition of cell proliferation and apoptosis directly linked to inhibition of Akt and has shown potent antitumor activity in vivo in several xenograft models. See, e.g., Mulvihill et al., “Discovery of OSI-906: a selective and orally efficacious dual inhibitor of the IGF-1 receptor and insulin receptor”, Future Medicinal Chemistry, 1(6):1153-71 (2009).

Both IGF-1R and IR are transmembrane receptor kinases that serve as key regulators of the IGF signaling axis. Activation of IGF-1R and IR (e.g., in response to stimulatory ligands) results in proliferative and anti-apoptotic effects via the MAPK and PI3K/Akt pathways. Activation of IGF-1R and IR also directly affects glucose metabolism by decreasing gluconeogenesis, increasing glycogen formation, and increasing translocation of GLUT transporters, thereby increasing overall glucose uptake in tissues. Dysregulated IGF-1R/IR signaling has been implicated in many human cancers and has been implicated in resistance to anti-cancer therapies, including cytotoxic chemotherapy, hormonal agents, and radiation therapy. IGF-1R/IR overexpression has been demonstrated in several tumor types, including breast, prostate, lung, ovarian, and colon cancers. Moreover, increased activation and overexpression of IGF-1R/IR is associated with an increased propensity for invasion and metastasis, and this reduces the chances of survival of the patient.

IR is a transmembrane receptor tyrosine kinase receptor, which plays an important role in glucose homeostasis in higher organisms. The structure of the IR was reported in 2006. Current binding models indicate that there are two binding sites on the insulin receptor, namely, two different low-affinity ligand-binding regions. The receptor consists of two α and two β subunits linked by disulfide bonds. Binding, results in structural changes in the receptor, which affords cross-linking of the bound ligand from the low-affinity site (Site 1) to the second site (Site 2). The current models also indicate that there are two binding sites on the surface of insulin, which afford binding with the two sites on the receptor. The preceding discussion outlines how tyrosinse kinase receptors, including IR, mediate their activity and potentially retard tumorigenesis. See, e.g., McKern et al., “Structure of the insulin receptor ectodomain reveals a foldedover conformation”, Nature, 443, 218-221 (2006).

IGF-1R is a tetrameric transmembrane receptor tyrosine kinase that is widely expressed in normal human tissues, and is up-regulated in a number of human cancers, including colorectal, non-small cell lung, ovarian and pediatric cancers. The receptor is composed of two α and two β subunits linked by disulfide bonds in which the extracellular a subunit is responsible for ligand binding and the β subunit consists of a transmembrane domain and a cytoplasmic tyrosine kinase domain. Ligand binding activates the tyrosine kinase activity of IGF-1R and results in trans-β subunit autophosphorylation and stimulation of signaling cascades that include IRS-1/phosphatidylinosiol 3-kinase/AKT/mTor targets of rapamycin and growth factor receptor binding protein 2/Sos/Ras/mitogen-activated protein kinase pathways. Activation of IGF-1R has been reported to stimulate proliferation, survival, transformation, metastasis, and angiogenesis, whereas inhibition of IGF-1R has been shown to retard tumorigenesis in several human xenograft models. Increased expression of IGF-1R and its cognate ligands, IGF-I and IGF-II, has been observed in a wide range of solid tumors and hematologic neoplasias relative to normal tissue levels. Epidemiologic studies have shown an increased risk for the development of colon, lung, breast and bladder cancers with increased circulating levels of IGF-1R. Additionally, IGF-1R expression level has been correlated to poor prognosis in renal cell carcinoma. The IGF-1R signaling mechanism has also been linked to resistance to various anti-tumor therapies, including epidermal growth factor receptor inhibitors. See, e.g., Reidemann et al., “IGF1R Signaling and its Inhibition”, Endocrine-Related Cancer, 13, S33-S43 (2006); LeRoith et al., “The Insulin-like Growth Factor System and Cancer, Cancer Letters, 195, 127-137 (2003); Baserga et al., “The IGF-1 Receptor in Cancer Biology”, Int. J. Cancer, 107, 873-877 (2003); and Ji Q., et al., “A Novel, Potent and Selective Insulin-like Growth Factor-1 Receptor Kinase Inhibitor Blocks Insulin-like Growth Factor-1 Signaling in vitro and Inhibits Insulin-like Growth Factor-1 Receptor-Dependent Tumor Growth in vivo”, Mol. Cancer. Ther., 6, 2158-2167 (2007).

A signaling pathway particularly considered herein as a target downstream of IGF-1R and IR is the PI3K signaling pathway. The PI3K signaling pathway is linked to both growth control and glucose metabolism. This pathway directly regulates glucose uptake and metabolism via Akt-mediated regulation of glucose transporter activation and expression (GLUT1 and GLUT4), enhanced glucose capture by increased hexokinase activity, and stimulation of phosphofructokinase activity. See, e.g., Elstrom, R., et al., “Akt Stimulates Aerobic Glycolysis in Cancer Cells”, Cancer Res., 64, 3892-3899 (2004); Clemmons D., “Involvement of Insulin-like Growth Factor-1 in the Control of Glucose Homeostasis”, Current Opinion in Pharmacology, 6, 620-625 (2006); DeBerardinis R., et al., “Brick by Brick: Metabolism and Tumor Cell Growth”, Current Opinion in Genetics & Development, 18, 54-61 (2008); and Vander Heiden M., et al., “Understanding the Warburg Effect The Metabolic Requirements of Cell Proliferation”, Science, 324, 1029-1033 (2009).

PI3K activation thus renders the cells dependent on glucose, thereby leading to glucose addiction. Small molecules that disrupt this pathway lead to decreased glucose uptake. This ability to inhibit glucose uptake correlates well with tumor regression in a number of cancers. Glucose withdrawal can induce cell death in a manner indistinguishable from that observed upon withdrawal of growth factor signaling.

Glucose serves as the fundamental energy source for all eukaryotic cells. Normal cells rely on a process called respiration (mitochondrial oxidative phosphorylation) for the majority of their energy needs. In this pathway the TCA or Krebs cycle utilizes pyruvate formed during glycolysis resulting in 36 ATP molecules per molecule of glucose. However, cancer cells differ in that they typically depend more on glycolysis or the anerobic breakdown of glucose for their source of ATP. This increase in glycolysis, even in the presence of oxygen (aerobic glycolysis), has been termed the Warburg Effect, as first described by Otto Warburg more than 80 years ago (Kim J and Dang C, (2006) Cancer's Molecular Sweet Tooth and the Warburg Effect, Cancer Res, 66, 8927-8930); Garber K, (2006) Energy Deregulation: Licensing Tumors to Grow, Science, 312, 1158-1159). Though the process of aerobic glycolysis is far less efficient, generating only two ATP molecules per glucose molecule, it appears to confer an advantage for survival of cancer cells. It has only been recently proposed that the metabolism of cancer cells has been adapted to facilitate the uptake and incorporation of nutrients such as nucleotides, amino acids and lipids to increase the biomass of the cell leading to proliferation (Vander Heiden M et. al., (2009) Understanding the Warburg Effect: The Metabolic Requirements of Cell Proliferation, Science, 324, 1029-1033).

In support of this are recent studies linking several signaling pathways, particularly PI3K (phosphoinositide 3-kinase) and AKT in cell proliferation that are also involved in regulating cellular metabolism (Elstrom R, et. al., (2004) AKT Stimulates Aerobic Glycolysis in Cancer Cells, Cancer Res, 64, 3892-3899). The PI3K signaling pathway has been strongly implicated in cell growth directing amino acids into protein via mTOR. Additionally, this pathway also regulates glucose metabolism by affecting uptake and utilization directly by signaling of downstream AKT which regulates glucose transporter expression, hexokinase activity and stimulation of phosphofructokinase activity (Hammerman P et. al., (2004), “Beginnings of a Signal-Transduction Pathway for Bioenergetic Control of Cell Survival”, Trends in Biochemical Sciences, 29 (11), 586-591); Tong X et al. (2009) “The Molecular Determinants of de novo Nucleotide Biosynthesis in Cancer Cells”, Current Opinion in Genetics & Development, 19, 32-37); Vander Heiden M et. al., (2009) Understanding the Warburg Effect: The Metabolic Requirements of Cell Proliferation, Science, 324, 1029-1033.) Thus, activation of the PI3K pathway renders cells to become dependent on a high flux of glucose. Disruption of this pathway leads to decreased glucose consumption in tumors which can then be monitored by [¹⁸F]-FDG PET studies. Glucose withdrawal can induce cell death that is essentially indistinguishable from growth factor withdrawal (apoptosis). Therefore, the ability to measure a response of a therapeutic agent that disrupts glucose metabolism can be predicted through the use of FDG-PET. In fact, in cancer patients, response to therapy is predicted by the ability of agents to disrupt glucose metabolism as measured by uptake of FDG-PET. See, e.g., Kelloff G., et al., “Progress and Promise of FDG-PET Imaging for Cancer Patient Management and Oncological Drug Development”, Clin. Cancer Res., 11 (8), 2785-2808 (2005); Mankoff D., et al., “Tumor-Specific Positron Emission Tomography Imaging in Patients: [¹⁸F] Fluorodeoxyglucose and Beyond”, Clin. Cancer Res., 13 (12) 3460-3469 (2007); and Vander Heiden, et al., Science, 2009 (Ibid.).

The major direct effect of the IGF-1R/IR axes to regulate cell-cycle progression is exerted at the G₁-S interface which is mediated through activation of both the PI3K and AKT pathways (Samani A., et al., (2009) The Role of the IGF System in Cancer Growth and Metastasis: Overview and Recent Insights, Endocrine Reviews, 28 (1), 20-47). Therefore, tyrosine kinase inhibitors that disrupt these axes will result in altered glycolysis, which should be identified by the use of FDG-PET as a rapid PD biomarker of this change in cellular metabolism.

In normal cells, activation of PI3K is highly controlled by dephosphorylation of phosphatidylinositol by the phosphatase and tensin homolog (PTEN) protein. However, in cancers, activation of this pathway can be augmented by a variety of mechanisms, which together constitute one of the most prevalent classes of mutations in human malignancy (e.g. PI3CA, AKT2, PTEN, BCR-ABL, HER2/neu). Regardless of the mutation, activation of Akt is likely the most important signaling event in relation to cellular metabolism, because Akt is sufficient to drive glycolysis and lactate formation and suppress macromolecular degradation in cancer (DeBeardinis R., et al., “The Biology of Cancer: Metabolic Reprogramming Fuels Cell Growth and Proliferation”, Cell Metabolism, 7, 11-20 (2008); and Kroemer G., “Tumor Cell Metabolism: Cancer's Achilles' Heel”, Cancer Cell, 13, 472-482 (2008)).

The cancer can be any type of cancer, and typically, a cancer observable by PET, such as a cancer that has evidenced itself as a growth in biological tissue. The cancer can be, for example, a cancer of the lung (e.g., non-small cell lung), breast, prostate, colon, colorectal, ovaries, kidney, bladder, lymph node (e.g., Hodgkin's disease or non-Hodgkin's lymphoma), stomach, brain, or a pediatric cancer. The cancer particularly considered herein is in the form of a tumor. The tumor can be, for example, a melanoma, myeloma, carcinoma, sarcoma, or blastoma.

It has been shown that PET (e.g., FDG-PET) can be used as an effective means for quickly and non-invasively monitoring PD endpoints and other characteristics, as well as for predicting treatment response to a RTK-targeting drug, particularly an IGF-1R or IR inhibiting drug, such as OSI-906. Both in vitro and in vivo results have been presented herein as exemplary evidence of the efficacy of this method. For example, in vitro results have been presented showing that the IGF-1R/IR positive cell line, H292, responded rapidly to OSI-906 treatment by significantly reducing uptake of ³H-2-deoxyglucose. Glucose (radiotracer) uptake was inhibited in a dose dependent manner and occurred within minutes of drug treatment correlating with inhibition of IGF-1R/IR and other downstream signaling events (e.g. Akt). Furthermore, in vivo xenografts bearing the same cell line demonstrated significantly reduced uptake of ¹⁸F-FDG at 2 and 4 hours post treatment, which correlated with substantial (>80%) target inhibition of both phospho-IGF1-R and phospho-IR in the tumor lysates. Target inhibition of phospho-IGF1-R and phospho-IR was confirmed in the tumor lysates by RTK array analysis. Western blot analysis of in vivo tumor lysates confirmed significant inhibition of phospho-AKT and other markers associated with altered glycolysis. In contrast, in the IGF-1R/IR negative H441 xenograft model, no significant change in uptake of ¹⁸F-FDG was observed over the same time course. Thus, evidence has been presented showing that identification of active doses, as well as the efficacy and suitability of an RTK-targeting drug for a patient, can be assessed and quantified by PET in the preliminary or planning stages of treatment.

In some aspects, the method begins with administration of the anti-cancer drug into a subject suspected or known to be suffering from a cancerous condition. In further aspects, the dosage of the drug is a therapeutic dose. In further aspects, the dosage of the drug is a non-therapeutic dose. A non-therapeutic dose can be preferred, for example, when it is desired to prevent the patient from being subjected to one or more side-effects that can occur at a therapeutic dose, while the non-therapeutic dose is capable of providing (i.e., by PET imaging) pertinent information related to the PD effects of the drug. The amount of the drug administered should, however, be at least an amount capable of eliciting a pharmacological response of the suspect tissue.

The drug can be administered to the patient in any suitable manner that can allow the drug to enter the bloodstream. Typically, the drug is administered by injection (e.g., intravenously or intramuscularly). However, the drug may also be administered orally (e.g., enterally), parentally (e.g., by infusion through the skin), or topically (e.g., on the skin), if the drug can effectively and efficiently enter the bloodstream by these modes. For oral administration, liquid or solid oral formulations can be given. These include, for example, tablets, capsules, pills, troches, elixirs, suspensions, and syrups.

Further, the radionuclide-labeled glucose analogue is administered prior to administration of the drug. Subsequent monitoring of a glucose uptake response of a solid tumor or cancerous tissue by positron emission tomography imaging of said solid tumor or cancerous tissue is utilized in order to establish a “baseline” or normalized level of glucose uptake. Further still, the baseline of the glucose uptake response is compared to the subsequent glucose uptake response after administration of the drug and the radionuclide-labeled glucose analogue.

The radionuclide in the labeled glucose analogue is a radionuclide observable by PET (i.e., that renders the labeled glucose a PET tracer). Some examples of suitable radionuclides for PET include fluorine-18, oxygen-15, nitrogen-13, carbon-11, and iodine-124. The labeled glucose analogue can be any labeled glucose analogue that can be absorbed (i.e., taken up) in cells of biological tissue, particularly cancerous tissue. In a particular embodiment, the labeled glucose analogue is a deoxyglucose analog. In a particular embodiment, the labeled glucose analogue is ¹⁸F-FDG.

The labeled glucose is administered in a diagnostic-effective amount. A diagnostic-effective amount is an amount that provides sufficient uptake by target cells such that glucose uptake by the target cells is visible by PET. The amount of labeled glucose administered depends on several factors, including the weight of the subject and the type of radionuclide label being used. Typically, a diagnostic-effective amount corresponds to a dosage corresponding to at least about 15, 20, 25, or 30 mCi per patient injection, or a range therein.

Following administration of the labeled glucose, the subject (or specifically, the tissue under study) is scanned (i.e., imaged) about 0.5, 1, 2, 3, 4, or 24 hours from administration of the labeled glucose by use of a PET technique. In other aspects, the subject may be serially imaged over several days to evaluate treatment. The PET technique can be conducted in accordance with any of the PET methodologies known in the art, as treated in, for example, Weber W. A., “Positron Emission Tomography as an Imaging Biomarker”, J. Clin Oncology, 24 (10) 3282-3292 (2005), and Mankoff D. A., et al., “Tumor-Specific Positron Emission Tomography Imaging in Patients: [¹⁸F]Fluorodeoxyglucose and Beyond”, Clin. Cancer Res., 13 (12), 3460-3467 (2007). The PET scan can be augmented or combined with any related imaging technique known in the art, such as, for example, x-ray computed tomography (CT), magnetic resonance imaging (MRI), functional magnetic resonance imaging (fMRI), ultrasound, and single photon emission computed tomography (SPECT).

PET imaging is used to determine the level of glucose uptake in the tissue under study. Typically, as is well known in the art, the glucose uptake is expressed as a standardized uptake value (SUV).

In a particular aspect, the observed glucose uptake is used for determining the efficacy of the drug in inhibiting the target of interest and eliciting a pharmacological response that could include subsequent inhibition of growth or sustainment of cancerous tissue. For example, the results of the PET imaging procedure can function as a pre-trial run (i.e., preclinical trial) of the drug for a patient. The pre-trial run is typically useful for optimizing future treatment of a patient, establish or refine a diagnosis, or for patient selection. Often, for this purpose, the drug is administered at a non-therapeutically effective dose. This lower dosage level, also known as a “sub-efficacious dose,” can serve, by PET imaging, to provide information on the pharmacological and PD activity of the drug for a particular patient. The resulting information is useful, for example, for identifying tumors/patients sensitive for a particular drug (i.e. responders or non-responders), pharmacologically active drug concentrations, or to obtain a biodistribution profile of the drug. The information gathered can also be useful in determining whether the drug can sufficiently distinguish target cells from non-target cells for a particular patient, and thereby may predict side effects. The specificity and efficacy of the drug is dependent on several factors, including the type of cancer and the unique biochemical receptivity to the drug, which differs from patient to patient.

The information garnered from an initial PET scan of the PD effects of the drug can be used for determining a minimum-effective, or biologically effective dosage or dose range of the drug for a particular patient. In this way, a customized drug treatment plan can be provided for each patient. A significant advantage in using the pre-trial run is that it prevents a patient who is not responsive to, or otherwise would not benefit by regular dosage treatment of the drug, from unnecessarily receiving a regular dosage treatment or full regimen. In so doing, such a patient is safeguarded from the possible dangers of receiving a regular dosage treatment when such a treatment would either be too hazardous, ineffective, or unnecessary for the patient.

In one aspect, the drug is administered once in a single dose before glucose uptake is monitored at several time points during imaging with a PET technique. This method of analysis is particularly useful for determining the PD effects of the drug over time, which can be used for determining a proper dosage schedule. For example, depending on the glucose uptake results found over time for the dose of the drug, it may be determined that a certain dose of the drug be administered once every 48 hours, 24 hours, 12 hours, or 6 hours. The method may also be conducted by performing two or more administrations at varying concentrations of the drug, wherein each administration is followed by monitoring of the glucose uptake at one or more time points. Such a method can be used for determining an optimal dosage and dose schedule. In one aspect, the PET imaging is conducted continuously during the time that one or more administrations of the drug and/or labeled glucose is administered to the time that a change in glucose uptake is observed in targeted tissue. In another aspect, the PET imaging is conducted at particular times (i.e., as snapshots).

Generally, an inhibitory effect on the metabolic activity of the cancer cells is indicated by about a 10% drop in the glucose uptake value of the cancer cells. An inhibitory effect is more preferably evidenced by a drop of about 20%, 30%, 40%, or 50% in the glucose uptake value of the cancer cells.

In addition, if target tissue is found to be cancerous or pre-cancerous, the method can also be used to determine what mechanism (i.e., signaling pathway) is primarily responsible for the continued metabolic activity and survival (i.e., preservation and/or growth) of the cancerous tissue, and consequently, what drugs may be best suited for treatment. For example, multiple drugs, each with a different known mode of action, may be administered, wherein an observed change in the glucose uptake value elicited by one drug as compared to a smaller observed change caused by another drug indicates that the primary or dominant signaling pathway at work in the cancer cells is the one targeted by the drug that elicited a stronger response. Such information, in turn, can be used for determining an appropriate treatment strategy for the patient, e.g., prescribing to the patient a drug known to specifically target the signaling pathway found to be primarily responsible for metabolic growth and the survival of the cancer, or to benefit scientific research.

The drug administered in the method described herein may also be administered along with one or more other drugs that may augment or modulate the ability of the drug to inhibit glucose uptake in targeted tissue. The augmenting or modulating co-drug considered herein can be, for example, any anti-cancer drug, including a RTK-inhibiting drugs or chemotherapeutics. Some examples of possible co-drugs include erlotinib, sorafenib, imatinib, bosutinib, nilotinib, dasatinib, vinblastine, cisplatin, 5-fluorouracil, tamoxifen, and several others. The method may also include administration of an anti-angiogenesis agent. Some examples of such drugs include mTOR inhibitors (e.g. tensirolimus, sirolimus, OSI-027), MMP-2 (matrix-metalloproteinase 2) inhibitors, MMP-9 (matrix-metalloproteinase 9) inhibitors, VEGF (vascular endothelial growth factor) and VEGFR (vascular endothelial growth factor receptor) inhibitors (e.g. sunitinib, sorafenib, bevacizumab) and COX-II (cyclooxygenase II) inhibitors. Drugs that do not directly treat cancer but that have an adjuvant effect can also be included. Adjuvants include compounds that promote a cancer treatment mechanism or that ameliorate side effects.

### EXAMPLES

Examples have been set forth below for the purpose of illustration and to describe the best mode of the invention at the present time. However, the scope of this invention is not to be in any way limited by the examples set forth herein.

### Materials and Methods

OSI-906 can be prepared as disclosed in US 2006/0235031 (published Oct. 19, 2006) as Example 31 thereof.

¹⁸FDG was synthesized in the Vanderbilt University Medical Center Radiopharmacy and distributed by PETNET. The average radiochemical purity of the product was 98.5% and specific activity was >1,000 Ci/mmol.

The non-small cell lung carcinoma cell lines H292 and H441 were obtained form American Type Culture Collection (Manassas, Va.). These lines were maintained in RPM! 160 media (Cell Gro) supplemented with 10% FBS (Life Sciences) and 1% sodium pyruvate (Cell Gro) and maintained at 5.0% CO₂. Cells were grown to 90% confluency and then inoculated as noted.

³H-2-deoxyglucose Uptake Assay

H292 and H441 cells were seeded in 12 well microtiter plates (Becton Dickinson) at 9.0×10⁵ cells per well in normal glucose (11.1 mM) media for 6 hours (n=3 wells/group). The media was then changed to 5.5 mM glucose media and the cells were allowed to equilibrate overnight. On the day of the assay the media was again removed and replaced with 0.0 mM glucose (glucose starvation) for three hours. The cells were then co-administered OSI-906 (in a concentration ranging from 0.0 μM to 30 μM) and 0.15 μCi of ³H-2-deoxyglucose (Perkin Elmer). After a 5, 30, or 45-minute exposure the media was removed, the cells placed on ice and washed with ice-cold PBS buffer solution. The PBS was then removed and the cells were lysed in RIPA buffer for 15 minutes on ice. The lysates were harvested and counted in a Packard Liquid Scintillation counter. Uptake was calculated as raw counts and normalized to the 0.0 μM OSI-906 control wells.

### Xenograft Models

All animal studies were under the approval of the Institutional Animal Care and Use Committee in an American Association for Accreditation of Laboratory Animal Care-accredited vivarium and in accordance with the Institute of Laboratory Animal Research (Guide for Care and Use of Laboratory Animals, NIH, Bethesda, Md.). Mice were 8-week-old athymic nu/nu mice (Charles Rivers) and were housed in clean specific pathogen free rooms in groups of four mice per cage. The mice were allowed access to sterile rodent chow and water ad libitum except when undergoing FDG-PET imaging, as noted. The H292 and H441 cells were harvested from T225 flasks at 90% confluency and implanted in the right thigh subcutaneously at a concentration of 1.0×10⁷ cells per 100 μL of PBS inoculum. Palpable tumors were evident approximately 10 days post implant. When the tumors achieved a volume of approximately 300 mm³, the animals were sorted, randomized, and placed on study.

### ¹⁸F-FDG-PET Studies

Mice were fasted from food 12 hours prior to imaging with ¹⁸F-FDG (VUIIS Cyclotron). On the day of the study, the mice were administered 60 mg/kg of OSI-906 in 25 mM tartaric acid via oral gavage (n=8/group). After dosing, the mice were kept isolated in a chamber at a constant temperature of 30° C. using a warm air circulating device (Harvard Apparatus). At 2, 4, 8 and 24 hours post dosing of OSI-906, the mice were injected with 200 μCi (100 μl) of ¹⁸F-FDG via the retro orbital sinus. After 30 minutes, PET imaging was performed using a Concord small animal PET imager. Images were then collected and reconstructed. Analyses of separate regions of interest (ROI) drawn over the area of the tumors (and muscle) were conducted and glucose uptake was calculated as % injected dose per gram of tissue against known phantom standards.

### ¹⁸F-FDG Biodistribution Studies

Mice were fasted for 12-16 hours prior to dosing with the radiopharmaceutical. On the day of the study the mice were administered 60 mg/kg of OSI-906 in 25 mM tartaric acid via oral gavage (n=6/8 mice per group). Mice were kept isolated and under a warming lamp at a temperature of approximately 30° C. One hour before the desired sacrifice time, the mice were dosed with 4-6 μCi of ¹⁸F-FDG via the lateral tail vein. The mice were sacrificed 60 minutes later via CO₂ asphyxiation, and selected tissues removed, rinsed, weighed and then placed into scintillation tubes. The samples were counted using a Cobra II Scintillation Counter and the % injected dose per gram of tissue calculated against prepared standards.

### Western Blot Analysis

Assessment of the ability of OSI-906 to inhibit phosphorylation of IGF1-R and IR was analyzed from tumor lysates in vivo and cell lysates from in vitro glucose uptake assays by Western blotting. Additional markers of altered glycolysis included in this analysis were pAKT, pS6, pERK and AS160. Tumors were homogenized in 1 mL of TGH buffer per 200 mg of tissue on ice at 15,000 rpm until total homogenization was achieved. For analysis of IGF-1R and IR phosphorylation status, after pre-clearing by centrifugation (14,000 rpm/5 minutes), the lysate was incubated overnight at 4° C. with an antiphosphotyrosine antibody, and 30 μL Protein A-sephorase was then added and further incubated for an additional two hours. The immunoprecipitates were separated on SDS-PAGE and immunoblotted with a total IGF-1R antibody (Santa Cruz) and/or total IR antibody followed by detection using a HRP-conjugated goat anti-rabbit IgG antibody (Cell Signaling) with enhanced chemiluminesence. All other markers were assessed by conventional western blot analysis. Antibodies were obtained from Santa Cruz Biotechnology. Protein bands were imaged using a LAS-3000 Densitometer (Fujifilm) and quantified using Multi Gauge Software program (Fujifilm).

### RTK Analysis

Tumor lysates were prepared according to the manufacturer's protocol (Proteome Profiler, R&D Systems). Briefly, tumor lysates were solubilized in NP-40 lysis buffer and re-suspended for 30 minutes at 2° C. with rocking. This preparation was centrifuged at 14,000×g for 5 minutes and transferred to a clean tube. Protein concentration was then determined by BCA assay. The lysate was then incubated with the Human Phospho-RTK array at 1000 μg overnight at 2° C. with rocking. The arrays were then washed, and incubated with freshly prepared detection antibody for two hours. After washing, the arrays were exposed to film for 0.5-3.0 minutes. The developed arrays were then quantitfied using a Kodak scanner and image analysis software.

### Example 1

### Inhibition of ³H-2-deoxyglucose In Vitro

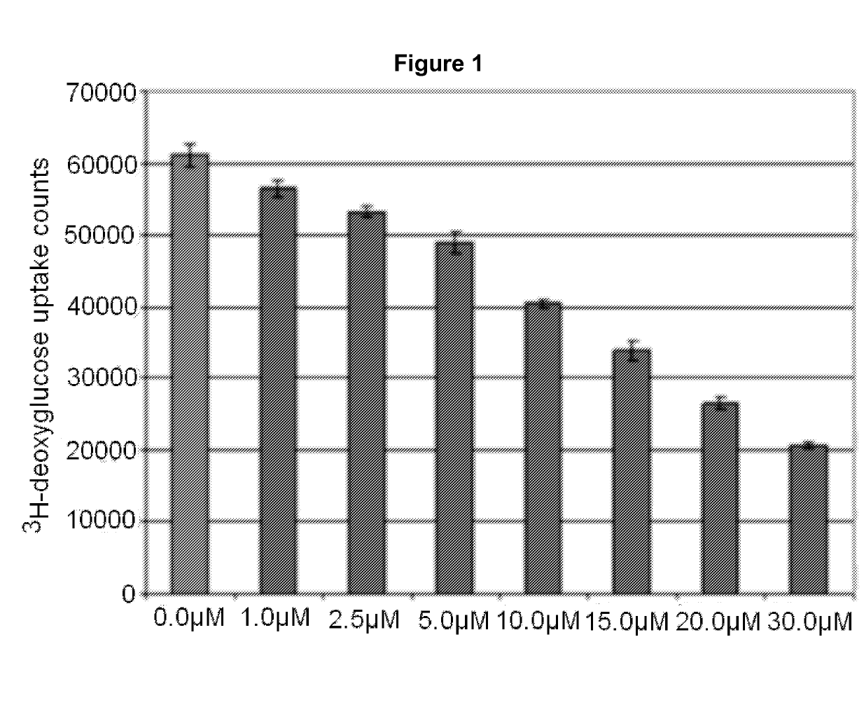

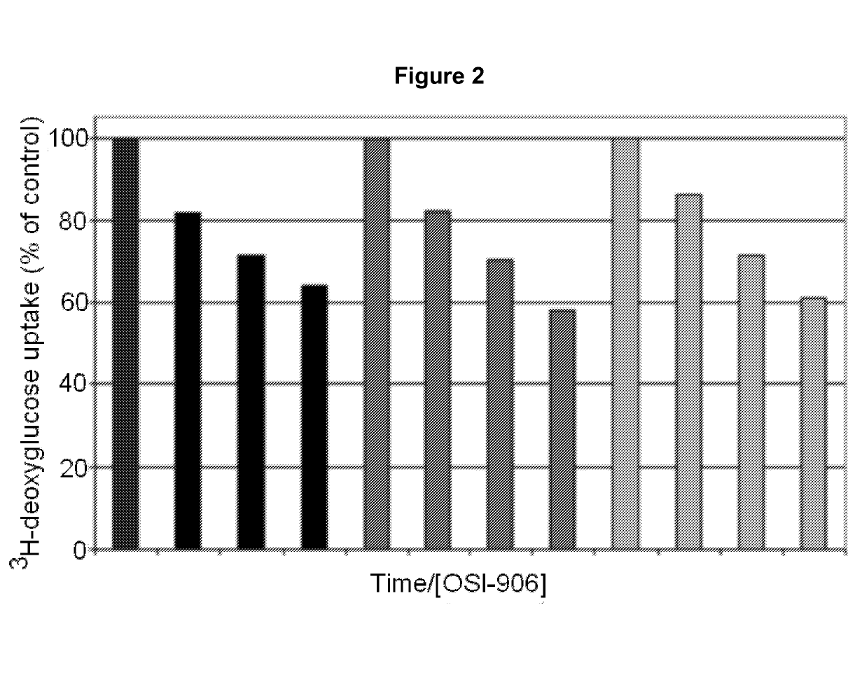

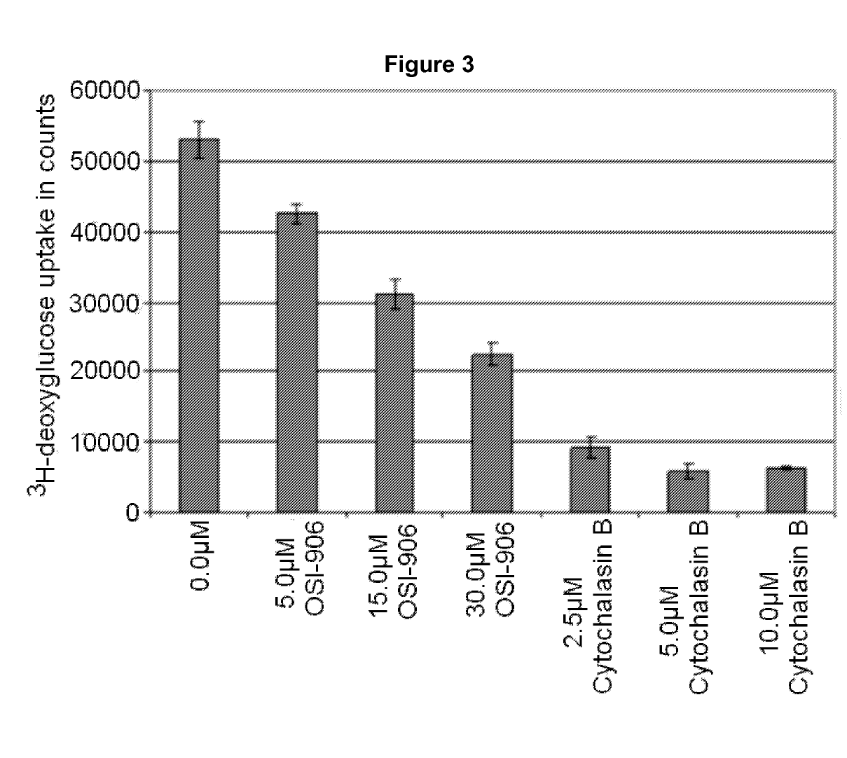

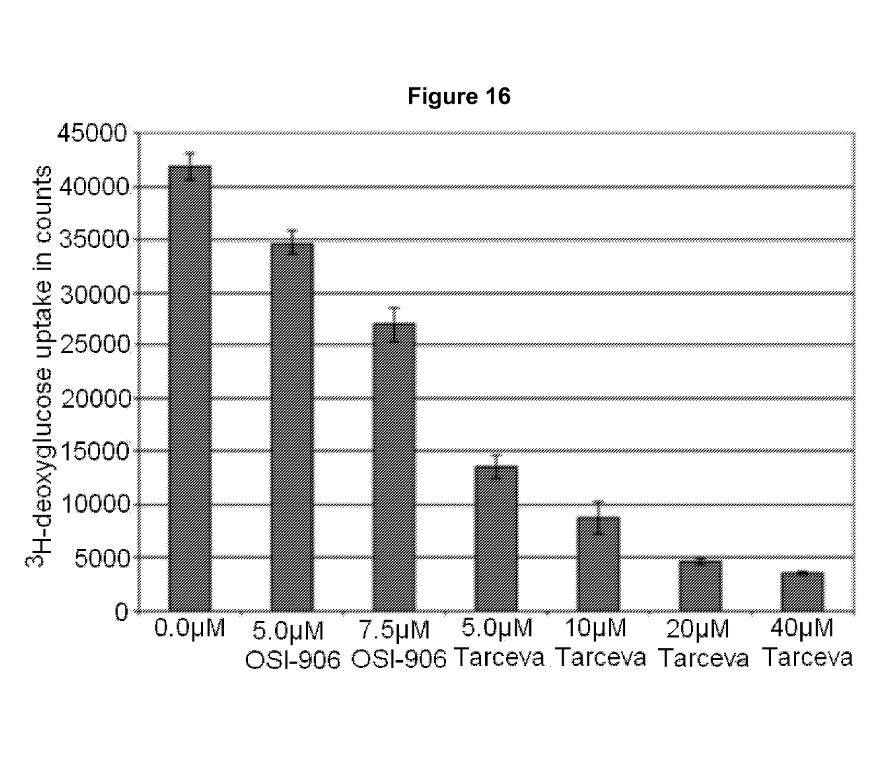

Uptake of ³H-2-deoxyglucose in H292 cells was inhibited in a dose dependent manner following incubation with OSI-906 (FIG. 1). The percent inhibition ranged from 12% to 60% as the dose increased from 1.0 μM to 30 μM OSI-906. Analysis for cell death by FACS analysis determined no significant cell death at all OSI-906 concentrations (1.0-30 μM) tested compared to 0.05% DMSO controls (data not shown). This inhibition of ³H-2-deoxyglucose uptake was determined to be rapid, with significant decrease noted as early as five minutes post treatment with increasing concentrations of OSI-906. FIG. 2 is a normalized histogram showing ³H-2-deoxyglucose uptake of H292 cells in vitro at 0.0 μM, 2.5 μM, 5.0 μM, and 7.5 μM concentration of OSI-906, measured at 45, 30 and 5 minutes post treatment. As a positive control, Cytochalasin B (2.5-10 μM) was administered to the H292 cells and evaluated for ³H-2-deoxyglucose uptake in an analogous manner (FIG. 3). FIG. 3 shows that Cytochalasin B significantly inhibits uptake of the radiotracer in this cell line, and that the inhibition of ³H-2-deoxyglucose by OSI-906 in H292 cells represents a rapid PD effect that may predict an altered uptake of ¹⁸F-FDG in an in vivo setting. FIG. 16 shows a histogram demonstrating rapid inhibition of ³H-2-deoxyglucose uptake in H292 cells in vitro after treatment with OSI-906 or erlotinib at selected concentrations of each agent.

### Example 2

### ¹⁸F-FDG-PET In Vivo Studies

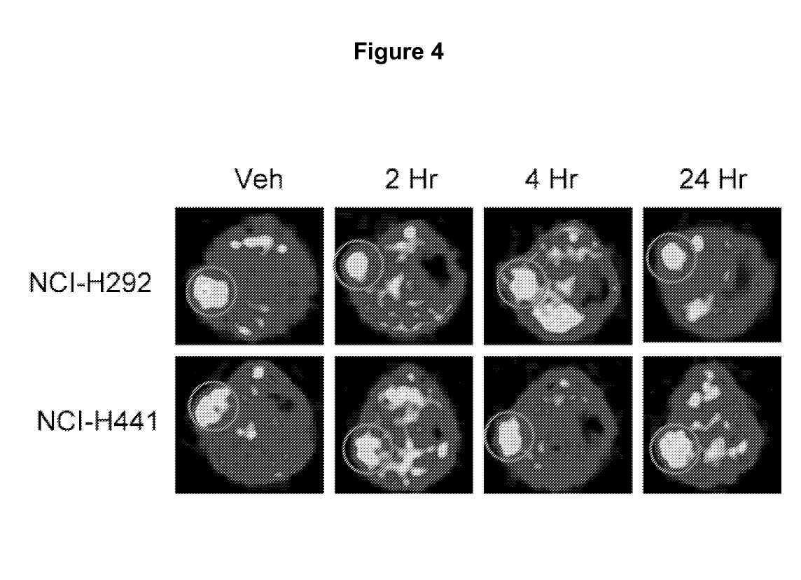

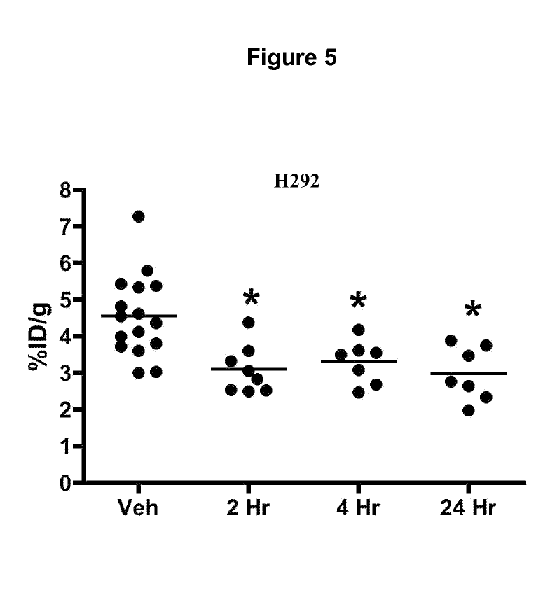

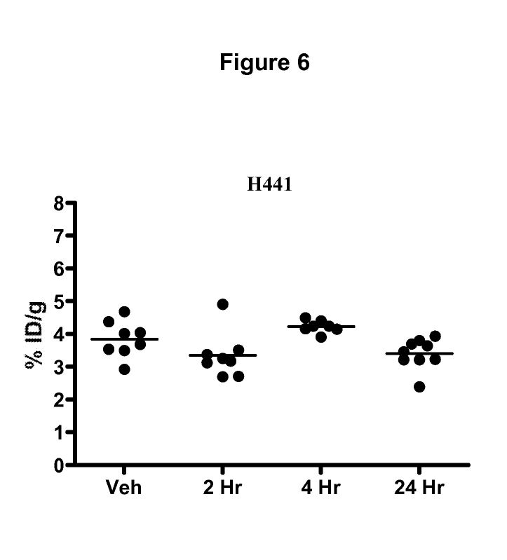

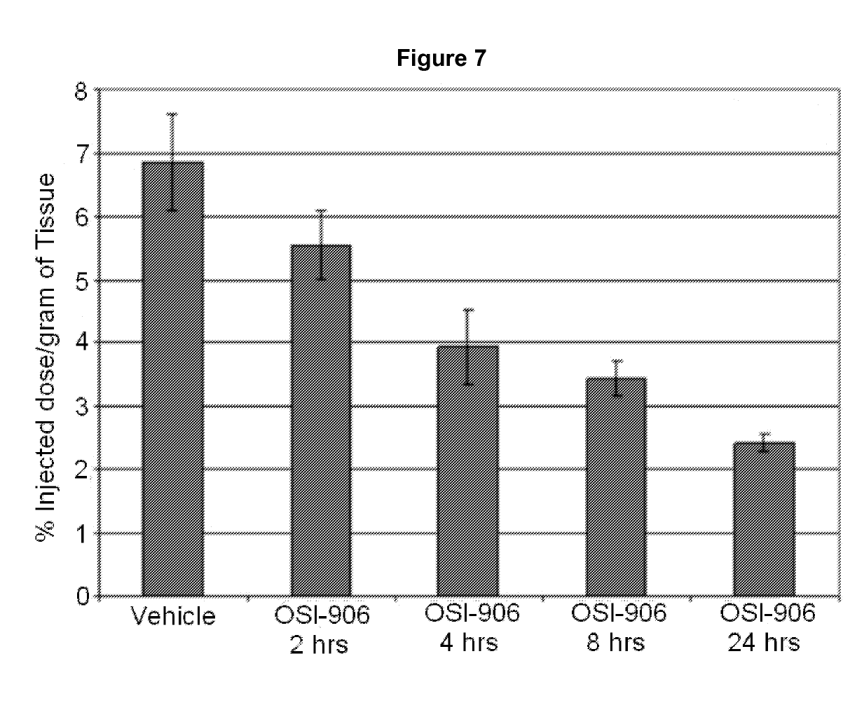

PET images of mice bearing the H292 and H441 xenografts are shown in FIG. 4. FIG. 4 shows the transverse and coronal serial images of the H292 xenografts and the transverse and coronal serial images of the H441 xenografts. Tumors have been encircled. The H292 xenografts (sensitive to OSI-906) show a significant decrease in ¹⁸F-FDG uptake at 2 and 4 hours post dosing with 051-906. By 24 hours, the level of uptake is similar to untreated control animals (data not shown). In contrast, the H441 xenografts (insensitive to OSI-906) do not demonstrate a significant change in uptake of ¹⁸F-FDG at any time point evaluated. These results are graphically shown in FIGS. 5 and 6. The decreased SUV in the H292 xenografts is suggestive of a rapid PD effect observed by ¹⁸F-FDG imaging. FIG. 7 shows quantitative assessment of the inhibition of [¹⁸F]-FDG uptake in tumor tissue as a function of OSI-906 drug treatment by biodistribution analysis. Similar to the decrease in uptake of ³H-2-deoxyglucose observed in vitro, H292 tumors also demonstrated a decrease in uptake of ¹⁸F-FDG compared to vehicle treated control animals. This decrease was observed as early as two hours post treatment with OSI-906, and continued throughout the time course of the study. Between 2 and 8 hours post OSI-906 treatment uptake in the tumor samples decreased by approximately 20-50% compared to the vehicle control animals.

### Example 3

### RTK Array Analysis

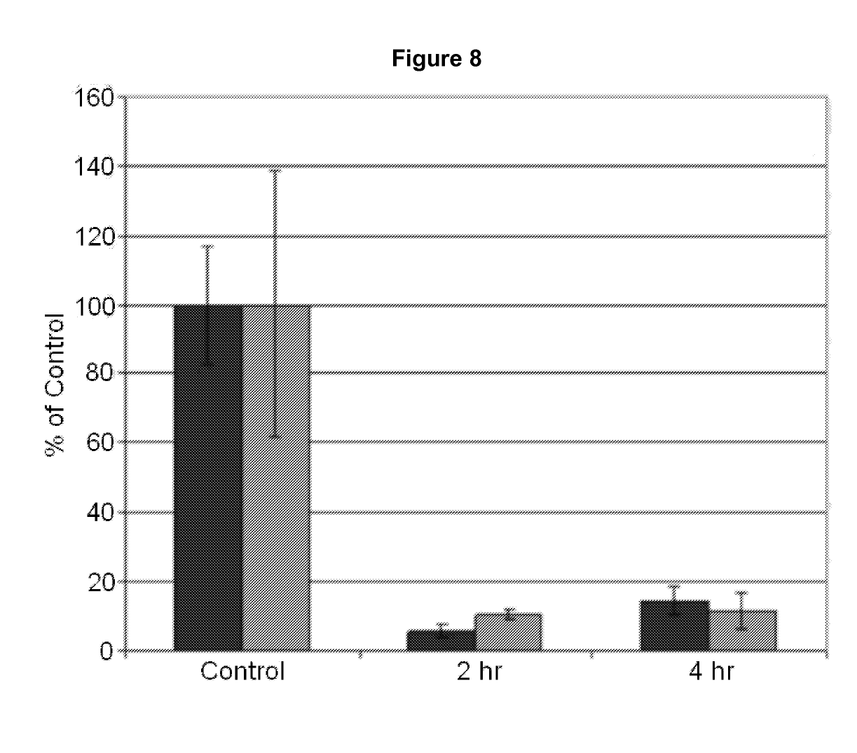

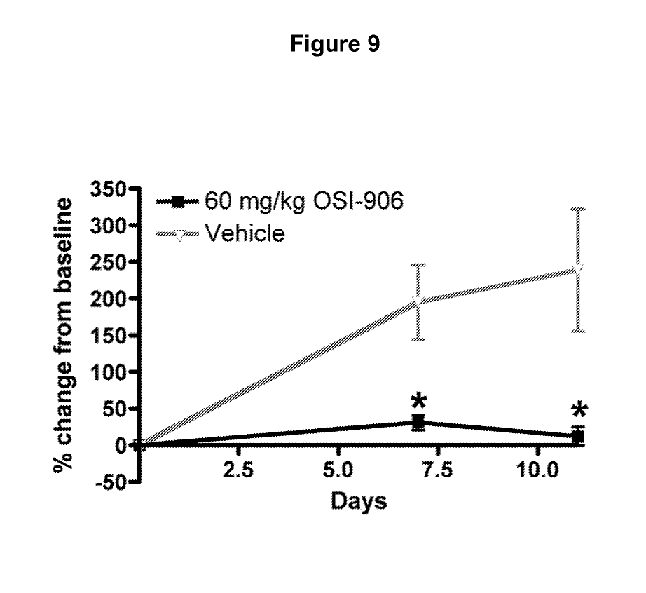

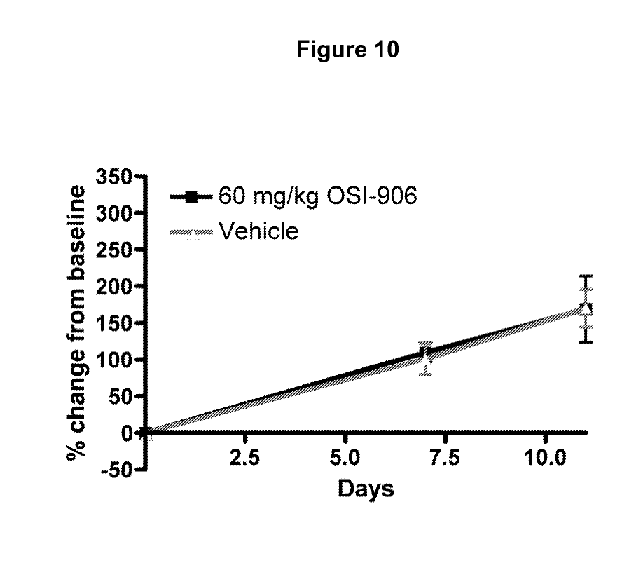

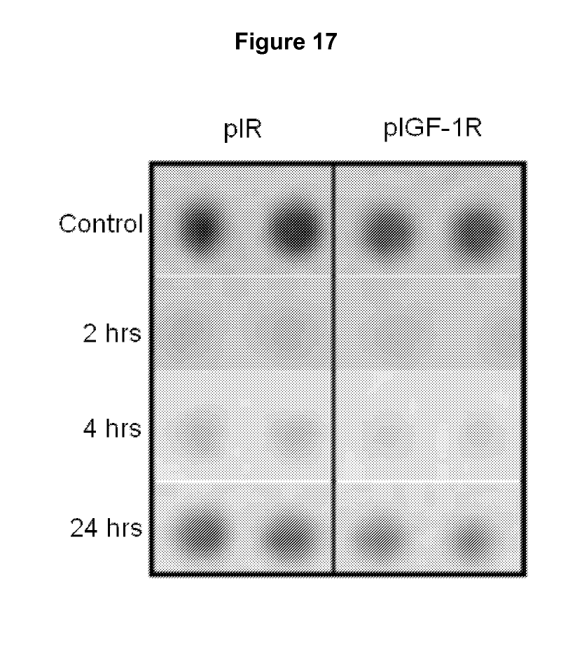

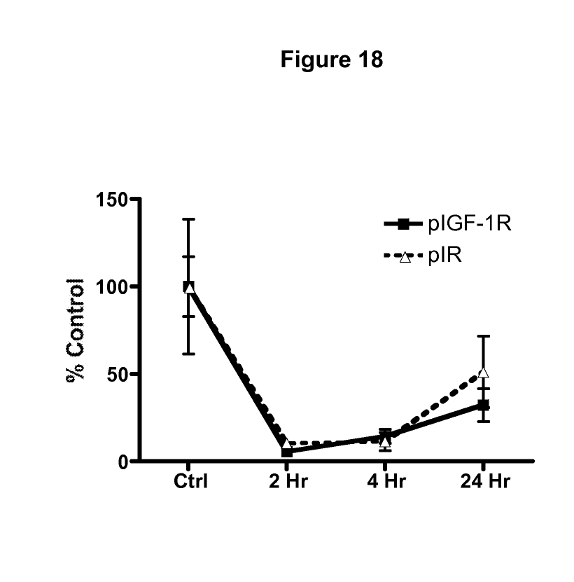

Target inhibition of phospho-IGF1-R and phospho-IR by a single dose of OSI-906 at 60 mg/kg in vivo is shown in FIGS. 8, 17, and 18. The data show that at 2 and 4 hours post treatment target inhibition of phospho-IGF1-R is approximately 85% of control returning to 70% of control by 24 hours. The effect on phospho-IR is less pronounced but data still demonstrates significant target inhibition of this receptor. Target inhibition of phospho-IR was 70% at 4 hours post treatment returning to 50% at 24 hours as shown in FIG. 18. Hence, pharmacodynamic effects (i.e. target inhibition) correlated with ¹⁸F-FDG uptake inhibition in vivo.

### Example 4

### Western Blot Analysis

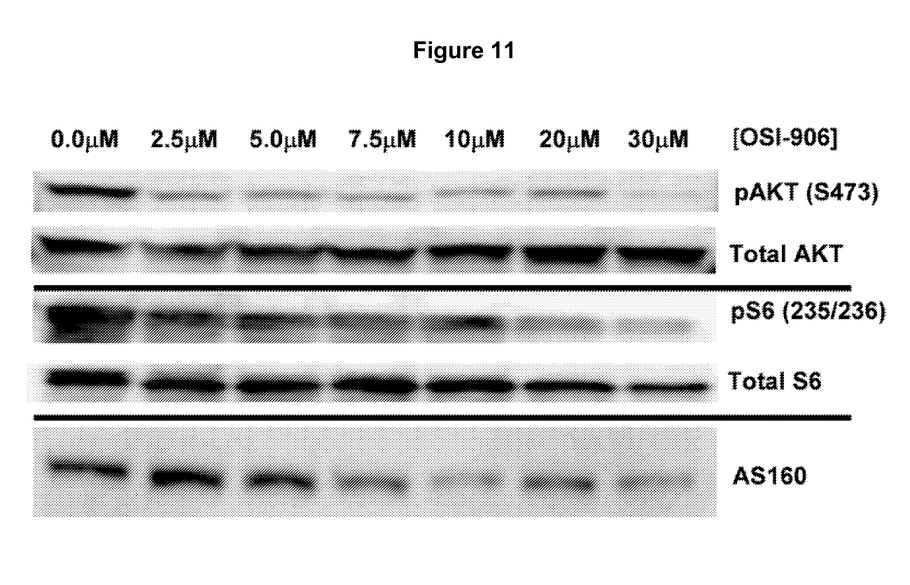

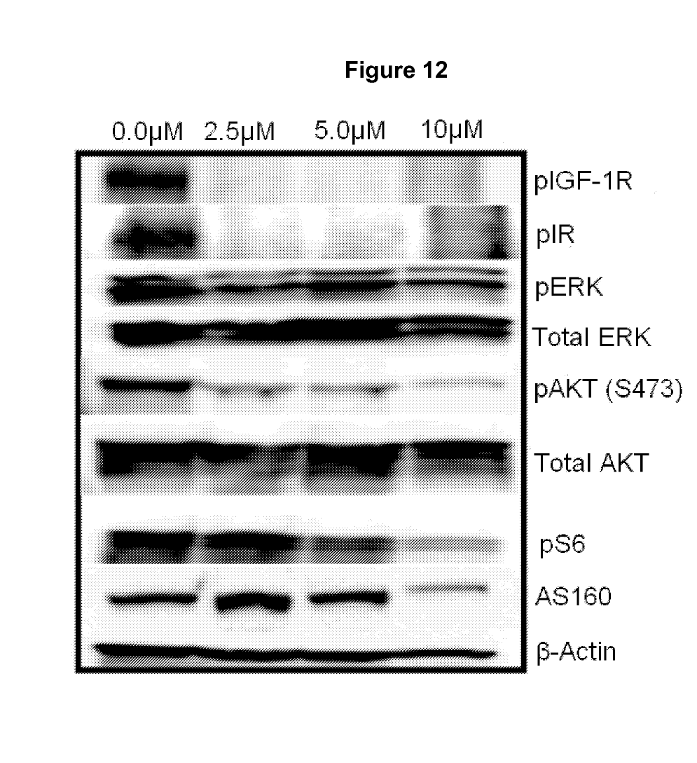

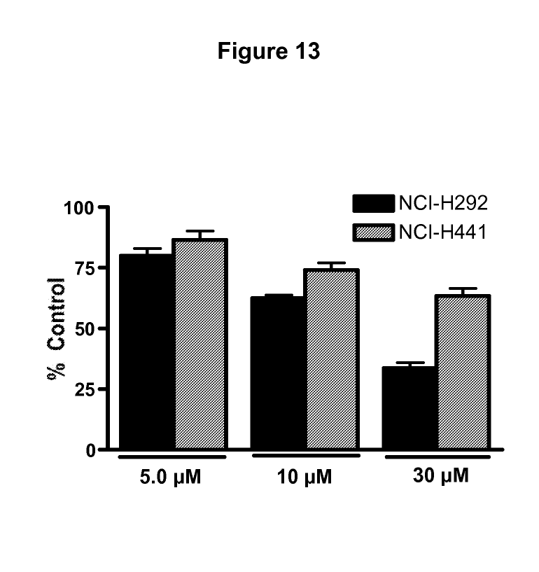

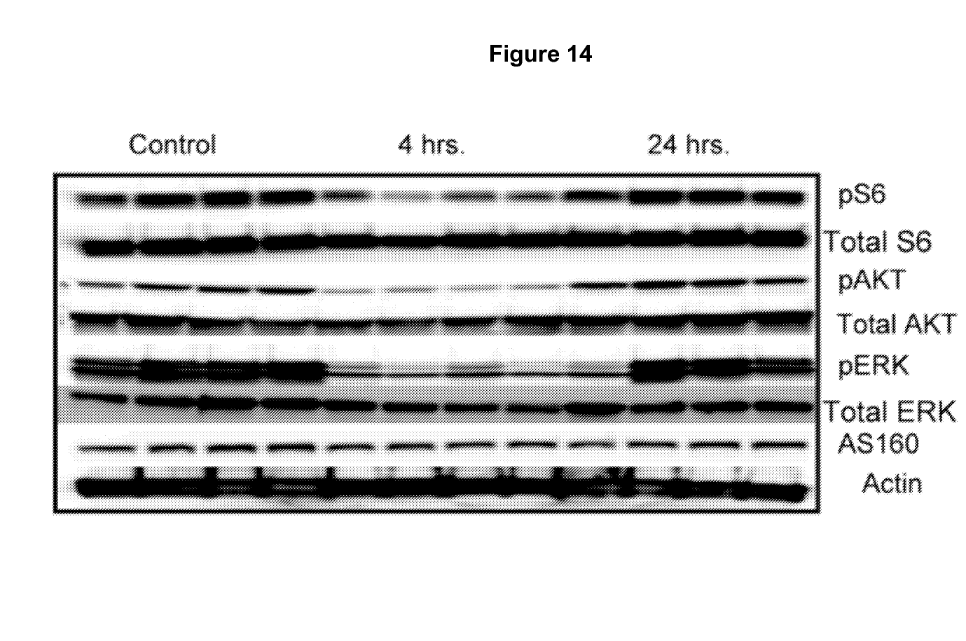

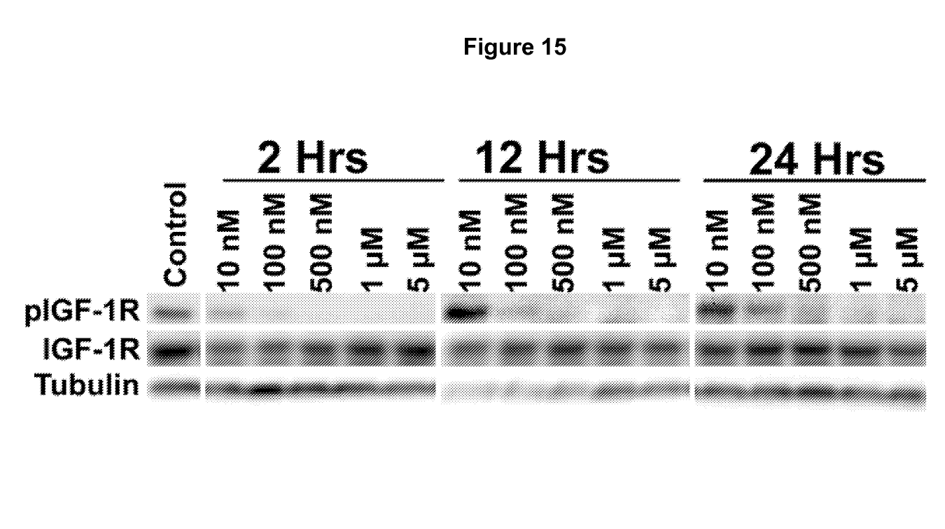

H292 in vitro cell lysates were treated with an increasing concentration of OSI-906 (0.0 μM-30 μM) and probed for phospho-AKT, phospho-S6 and AS160 as shown in FIG. 11. There is a dose dependent response with respect to decreased signal intensity in the bands for these proteins that are directly linked to altered glycolysis. FIG. 14 shows the results of a Western blot analysis from in vivo tumor lysates at selected time points from mice bearing the H292 xenografts that were treated with 60 mg/kg OSI-906 (n=4/group). The data clearly show decreased signal intensity for phospho-AKT, phospho-S6, phospho-ERK and AS160 after only four hours post treatment with OSI-906 compared to untreated control lysates.

### General Definitions and Abbreviations

Except where otherwise indicated, the following general conventions and definitions apply. Unless otherwise indicated herein, language and terms are to be given their broadest reasonable interpretation as understood by the skilled artisan. Any examples given are nonlimiting.

Any section headings or subheadings herein are for the reader's convenience and/or formal compliance and are non-limiting.

A recitation of a compound herein is open to and embraces any material or composition containing the recited compound (e.g., a composition containing a racemic mixture, tautomers, epimers, stereoisomers, impure mixtures, etc.). In that a salt, solvate, or hydrate, polymorph, or other complex of a compound includes the compound itself, a recitation of a compound embraces materials containing such forms. Isotopically labeled compounds are also encompassed except where specifically excluded. For example, hydrogen is not limited to hydrogen containing zero neutrons.

As used herein, the term ‘solvate’ is used herein to describe a molecular complex comprising the compound of the invention and one or more pharmaceutically acceptable solvent molecules, for example, ethanol.

As used herein, the term ‘hydrate’ is employed when the solvent is water.

As used herein, the phrase “elevated levels of insulin-like growth factor” refers to individuals with circulating levels of IGF-II in the upper quartile of the normal range.

As used herein, the term “pharmacodynamic effects (PD)” refers to one or more chemical, biological, or physiological effects of the drug on the patient, or on the biological tissue under study (e.g., cellular effects), particularly with respect to such effects that are observable using PET. A particular pharmacodynamic effect focused on herein is the effect of the drug in modulating or inhibiting cellular processes, particularly metabolic activity in suspect biological tissue, and particularly, metabolic activity as evidenced by glucose uptake in the tissue. Other pharmacodynamic effects include, for example, inhibition of cell proliferation or induction of apoptosis as measured by specific PET imaging probes.

As used herein, the term “rapid pharmacodynamic effect (rapid PD)” refers to one or more chemical, biological, or physiological effects of the drug on the patient, or on the biological tissue under study (i.e., cellular effects), particularly with respect to such effects that are observable using PET, either simultaneously or within 0.5-24 hours.

As used herein, the term, “therapeutically effective dose” refers to the amount of drug required to produce the desired effect of a drug.

As used herein, the term, “sub-therapeutic dose” refers to a dose of drug less than what is required to treat disease as prescribed on the label, which can be 10-95% of the therapeutic dose.

As used herein, the term, “IGF-1R kinase inhibitor” refers to any IGF-1R kinase inhibitor that is currently known in the art, and includes any chemical entity that, upon administration to a patient, results in inhibition of a biological activity specifically associated with activation of the IGF-1 receptor (e.g., in humans, the protein encoded by GeneID: 3480) in the patient, and resulting from the binding to IGF-1R of its natural ligand(s).

The following abbreviations are used:
min. minute(s)
h hour(s)
d day(s)
L liter
μL microliter
mg milligram
μg microgram
kg kilogram
mL milliliter
mmol millimole
μmol micromole
nM nanomolar
mM millimolar
μM micromolar
equiv. or eq. equivalents
μCi microcurie
mCi millicurie
wt % weight percentage

1. A method for determining whether to administer to a mammal a receptor tyrosine kinase-inhibiting drug for tumor treatment by determining a glucose uptake response in a tumor in the mammal, the method comprising:
(i) administering to said mammal a first amount of a radionuclide-labeled glucose analogue that is taken up into said tumor and functions as a tracer in positron emission tomography and thereby determining a first glucose uptake response;
(ii) administering to said mammal a test dosage of the drug;
(iii) administering to said mammal a second amount of the radionuclide-labeled glucose analogue for determining a second glucose uptake response;
(iv) determining the second glucose uptake response of said tumor by positron emission tomography imaging of said tumor at about 0.5-24 hours after completion of (ii); and
(v) determining whether to administer the drug based on its pharmacodynamic effect as determined by a degree of inhibition of the glucose uptake response observed by positron emission tomography imaging by comparing said first glucose uptake response and said second glucose uptake response, wherein an inhibitory effect on the glucose uptake response after administration of the test dosage indicates that the mammal is likely to benefit from treatment with the drug.

2. A method for treating a tumor of a mammal with a receptor tyrosine kinase-inhibiting drug comprising:
(A) initially determining if said mammal is likely to respond to treatment by:
(i) administering to said mammal a first amount of a radionuclide-labeled glucose analogue that is taken up into said tumor and functions as a tracer in positron emission tomography and thereby determining a first glucose uptake response;
(ii) administering to said mammal a test dosage of the drug;
(iii) administering to said mammal a second amount of the radionuclide-labeled glucose analogue for determining a second glucose uptake response;
(iv) determining the second glucose uptake response of said tumor by positron emission tomography imaging of said tumor at about 0.5-24 hours after completion of (ii); and
(v) determining an inhibitory effect as a degree of inhibition of the glucose uptake response observed by positron emission tomography imaging by comparing said first glucose uptake response and said second glucose uptake response; and
(B) administering to said mammal a therapeutically effective regimen comprising the drug if there is an inhibitory effect determined by (v).

3. A method for evaluating pharmacodynamic effects of a receptor tyrosine kinase-inhibiting drug in inhibiting glucose uptake in a tumor of a mammal, the method comprising:
(i) administering to said mammal a dosage of said drug;
(ii) administering to said mammal a radionuclide-labeled glucose analogue;
(iii) determining a glucose uptake response of said tumor by positron emission tomography imaging of said tumor at about 0.5-24 hours after completion of (i); and
(iv) determining a degree of inhibition of the glucose uptake response observed by positron emission tomography imaging.

4. The method according to claim 3, wherein said determining of (iii) is carried out at about 0.5 to 4 hours after completion of (i).

5. The method according to claim 3, wherein said determining of (iii) is carried out at about 0.5 to 2 hours after completion of (i).

6. The method according to claim 1, wherein said determining of (iv) is carried out at about 0.5 to 4 hours after completion of (ii).

7. The method according to claim 1, wherein said determining of (iv) is carried out at about 0.5 to 2 hours after completion of (ii).

8. The method according to claim 1, wherein said administration of (iii) is carried out before (ii).

9. The method according to claim 1, wherein said administration of (iii) is carried out simultaneously with (ii).

10. The method according to claim 1, wherein said administration of (iii) is carried out after (ii).

11. The method according to claim 1, wherein (ii) is carried out once and (iv) is carried out multiple times.

12. The method according to claim 1, wherein said test dosage is a sub-therapeutic dose.

13. The method according to claim 1, wherein said test dosage is a therapeutic dose.

14. The method according to claim 1, wherein said inhibitory effect is about 20% to 50%.

15. The method according to claim 1, wherein said method further comprises determining the responsiveness of said mammal to said drug based on said degree of inhibition of glucose uptake.

16. The method according to claim 1, wherein said method further comprises determining a suitable dosage or dosage range for said mammal based on said degree of inhibition of glucose uptake.

17. The method according to claim 1, wherein said method further comprises administering to said mammal varying concentrations of said drug to determine an optimal dosage or dosage range.

18. The method according to claim 1, wherein said method further comprises determining whether a level of glucose uptake in tissue of said mammal is indicative of said tissue being a tumor.

19. The method according to claim 1, wherein said method further comprises determining a signaling pathway primarily responsible for driving said tumor, and from this, determining an appropriate treatment strategy for said mammal.

20. The method according to claim 1, wherein said drug is a PI3K signaling pathway disruptor.

21. The method according to claim 1, wherein said drug is erlotinib, sunitinib, sorafenib, imatinib, bosutinib, nilotinib, or dasatinib.

22. The method according to claim 1, wherein said drug is an inhibitor of one or more of IR, IGF-1R or IR/IGF-1R heterodimer.

23. The method according to claim 1, wherein said drug is BMS-754807, AG538, AG1024, AMG-479, NVP-AEW541, figitumumab, R-1507, cixutumumab, dalotuzumab, IGF-1R antibodies, or OSI-906.

24. The method according to claim 1, wherein said drug is OSI-906.

25. The method according to claim 1, wherein said tumor is in a lung, breast, prostate, bladder, colon, rectum, pancreas, ovary, stomach, head, neck, esophagus, liver, adrenal gland, or kidney of said mammal.

26. The method according to claim 1, wherein said tumor is in a lung of said mammal.

27. The method according to claim 1, wherein said tumor is NSCLC.

28. The method according to claim 1, wherein said mammal is human.

29. The method according to claim 1, wherein said radionuclide-labeled glucose analogue is ¹⁸F-FDG.

30. The method according to claim 2, wherein said drug is OSI-906.

31. The method according to any one of claim 3, wherein said drug is OSI-906.
